(19) United States
(12) Patent Application Publication
(10) Pub. No.: US 2016/0235389 A1
(43) Pub. Date: Aug. 18, 2016

(54) US IMAGING APPARATUS WITH AN ANTIBACTERIAL AND/OR AN ANTIMICROBIAL EMBEDDED IN OR ON A SURFACE THEREOF

(71) Applicant: B-K MEDICAL APS, Herlev (DK)

(72) Inventor: Morten Falk Reventlow, Frederiksberg (DK)

(21) Appl. No.: 14/624,669

(22) Filed: Feb. 18, 2015

(51) Int. Cl. A61B 8/00; C09D 5/14; A01N 59/20; A01N 25/10; A61B 8/14; A01N 59/16

(52) U.S. Cl. CPC A61B 8/4494; A61B 8/145; A61B 8/467; A61B 8/54; A61B 8/4405; A61B 8/4472; A61B 8/461; A01N 59/16; A01N 59/20; A01N 25/10; C09D 5/14

(57) ABSTRACT

An ultrasound imaging apparatus (100) includes a housing. The housing includes a plurality of sides (312-322, 412-422). The plurality of sides includes a material with at least one of an antibacterial or an antimicrobial agent thereon. The housing further includes an acoustic window (324, 424) of one of the sides. The housing further includes a transducer array (102) of transducing elements (104) disposed within the sides and adjacent to the acoustic window. In one instance, the housing is a housing of a probe (202), which is separate and distinct from a console that controls the probe and processes received data. In another instance, the housing is a housing of a single elongate hand held scanner (400), which includes components for controlling the probe and processing received data.

### TECHNICAL FIELD

The following generally relates to ultrasound imaging and more particularly to an ultrasound imaging apparatus with an antibacterial and/or antimicrobial embedded in and/or on a surface.

### BACKGROUND

Ultrasound imaging provides useful information about the interior characteristics of an object or subject such as a human or animal patient. Generally, an ultrasound imaging device includes at least a transducer array configured to emit ultrasound signals and receive echo signals. The transducer array is behind an acoustic window, which is placed in contact with a patient (e.g., directly or indirectly through an acoustic gel or the like) for an ultrasound procedure.

Where the patient has a contaminate such as pathogenic bacteria, a microbe, a fungi, etc., the ultrasound imaging device can become contaminated with the pathogenic bacteria, a microbe, a fungi, etc. Ultrasound imaging device are not single use devices. As such, the same ultrasound imaging device may be used with a plurality of different patients over the course of a day. As a consequence, the ultrasound imaging device can be a source of cross contamination between patients.

An approach to mitigating cross contamination between patients has been to clean the ultrasound imaging device between patients. For example, one approach is to manually clean at least a portion of the ultrasound imaging device that comes into contact with patients with a fabric or wipe that includes a cleaner, a disinfectant, etc. However, ultrasound imaging devices have lines, recesses, groves, pockets, etc. in which a contaminate can enter, and fabrics and wipes are not well-suited for removing contaminates from such areas.

Unfortunately, a pathogenic bacteria, a microbe, a fungi, etc. in such areas and/or elsewhere on a manually “cleaned” ultrasound imaging device can be a source of cross contamination between patients. In view of at least the above, there is an unresolved need for another(s) approach for mitigating cross contamination between patients.

### SUMMARY

Aspects of the application address the above matters, and others.

In one aspect, an ultrasound imaging apparatus includes a housing. The housing includes a plurality of sides. The plurality of sides includes a material with at least one of an antibacterial or an antimicrobial agent thereon. The housing further includes an acoustic window of one of the sides. The housing further includes a transducer array of transducing elements disposed within the sides and adjacent to the acoustic window.

In another aspect, an ultrasound imaging apparatus includes a probe with a surface with an antibacterial or an antimicrobial integrated therein, the probe enclosing a transducer array and including a first communications interface, wherein the surface of the probe includes at least one recess. The ultrasound imaging apparatus further includes a console, including: transmit circuitry configured to transmit control signals to the transducer elements, receive circuitry configured to receive echo signals to the transducer elements, a beamformer that processes the receive echo signals and produces ultrasound images, and a complementary communications interface.

In another aspect, a single enclosure hand held ultrasound imaging apparatus includes a housing with a surface having an antibacterial or an antimicrobial coating therein, the housing enclosing a transducer array, transmit circuitry, receive circuitry, a beamformer, a control and a display.

Those skilled in the art will recognize still other aspects of the present application upon reading and understanding the attached description.

### BRIEF DESCRIPTION OF THE DRAWINGS

The application is illustrated by way of example and not limited by the figures of the accompanying drawings, in which like references indicate similar elements and in which:

FIG. 1 schematically illustrates an example ultrasound imaging apparatus with a transducer enclosed in a housing with an antibacterial and/or antimicrobial embedded in and/or on a surface thereof;

FIG. 2 schematically illustrates an example in which the housing is part of a probe that houses the transducer array and that connects to a separate console;

FIG. 3 schematically illustrates an example the probe;

FIGS. 4 and 5 schematically illustrate an example in which the ultrasound imaging apparatus is a single elongate hand held scanner; and

FIG. 6 schematically illustrates an example method.

### DETAILED DESCRIPTION

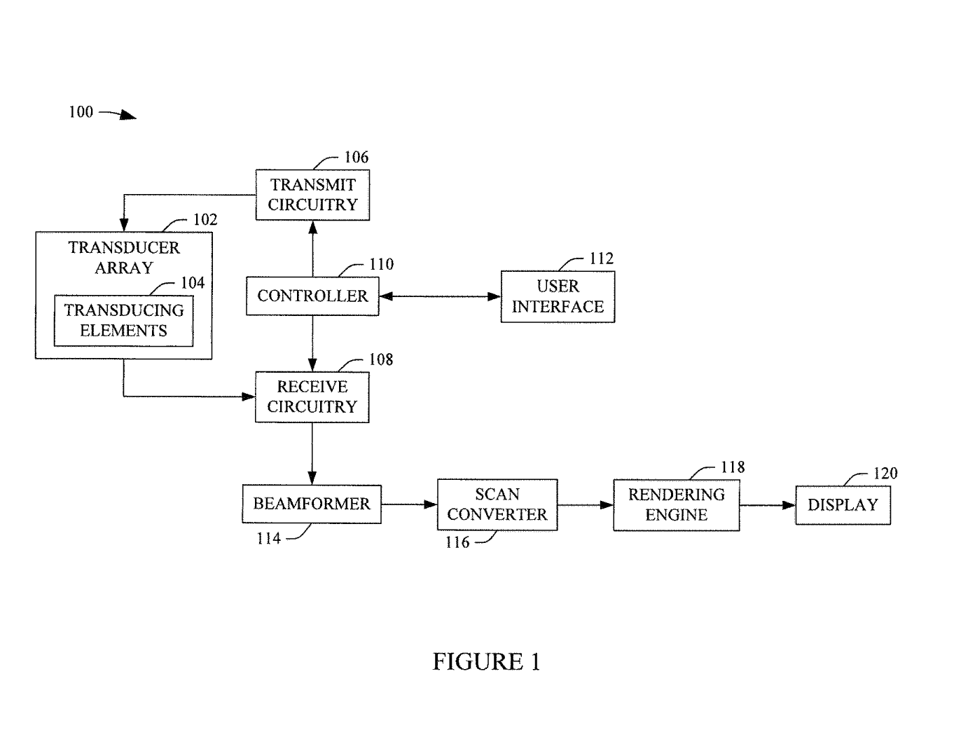

Initially referring to FIG. 1, an example ultrasound imaging apparatus 100 is illustrated.

As described in greater detail below, the ultrasound imaging apparatus 100 includes an antibacterial and/or antimicrobial material applied as an additive to a material of a housing and/or as a coating to a surfaces of the housing and/or other components of the apparatus 100. The antibacterial and/or antimicrobial material may facilitate mitigating cross contamination between patients by the ultrasound imaging apparatus 100.

The ultrasound imaging apparatus 100 includes a transducer array 102 with a one or two dimensional array of transducer elements 104. The transducer elements 104 are configured to transmit ultrasound signals and receive echo signals. Examples of suitable arrays include square, circular, irregular and/or rectangular arrays, linear, curved, etc., fully populated or sparse, etc.

The ultrasound imaging apparatus 100 further includes transmit circuitry 106 that generates a set of pulses that are conveyed to the transducer elements 104. The set of pulses actuates a corresponding set of the transducer elements 104, causing the transducer elements 104 to transmit ultrasound signals, e.g., into an examination or scan field of view.

The ultrasound imaging apparatus 100 further includes receive circuitry 108 that receives echoes generated in response to the transmitted ultrasound signals from the transducer elements 104. The echoes, generally, are a result of the interaction between the emitted ultrasound signals and the structure (e.g., tissue cells, flowing blood cells, etc.) in the scan field of view.

The ultrasound imaging apparatus 100 further includes a controller 110 that controls one or more of the transmit circuitry 106 or receive circuitry 108. Such control can include identifying and/or controlling at least one ultrasound imaging function such as selecting an imaging mode of operation (e.g., A-mode, B-mode, etc.), initiating scanning, etc. Such control can be based on an input indicative of a user selection.

The ultrasound imaging apparatus 100 further includes a user interface (UI) 112. The UI 112 includes one or more input devices (e.g., a button, a knob, a slider, a touch pad, etc.) and/or one or more output devices (e.g., a display screen, lights, a speaker, etc.). The user interface 112 can be used by a user to select an imaging mode of operation, initiate scanning, etc.

The ultrasound imaging apparatus 100 further includes beamformer 114 that processes the echoes, e.g., by applying time delays and weights to output signals of the channels and summing the time delayed and weighted signals, and/or otherwise beamforming received echoes. For B-mode, in one instance, the beamformer 114 generates a sequence of focused, coherent echo samples along focused scanlines of a scanplane.

The ultrasound imaging apparatus 100 further includes scan converter 116 that converts the output of the beamformer 114 (e.g., the sequence of focused, coherent echo samples along focused scanlines of a scanplane) to generate data for display, for example, by converting the data to the coordinate system of the display. The scan converter 116 can be configured to employ analog and/or digital scan converting techniques.

The ultrasound imaging apparatus 100 further includes a rendering engine 118 that visually presents the converted data via a display monitor 120. Such presentation can be in an interactive graphical user interface (GUI), which allows the user to selectively rotate, scale, and/or manipulate the displayed data. Such interaction can be through a mouse or the like, and/or a keyboard or the like, touch-screen controls and/or the like, and/or other known and/or approach for interacting with the GUI.

The beamformer 114, the scan converter 116 and/or other component of the ultrasound imaging apparatus 100 can be implemented via a processor (e.g., a microprocessor, central processing unit, etc.) executing one or more computer readable instructions encoded or embedded on a non-transitory computer readable storage medium such as physical memory. The processor can execute a computer readable instructions carried by a carrier wave, a signal, or other transitory medium.

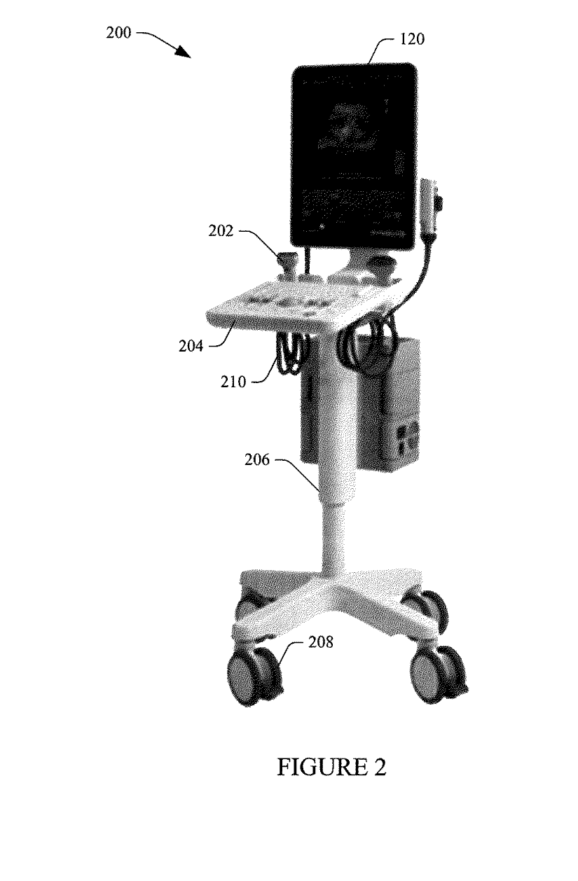

FIG. 2 illustrates an example configuration 200 of the ultrasound imaging apparatus 100 in which the transducer array 102 and the transducing elements 104 are housed in a probe 202 and the remaining components are located external to the probe 202. In this example, a console 204 houses the transmit and receive circuitry 106 and 108, the controller 110, at least a portion of the UI 112, the beamformer 114, the scan converter 116 and the rendering engine 118.

The console 204 and the display 120 are affixed to a mobile cart 206, which include movers 208 such as wheels, casters, etc. In another configuration, the cart 206 does not include movers, but instead is configured to rest on a table, desk, etc., attach to a wall, a support device, etc. The probe 202 electrically communicates with the console 204 through complementary interfaces via at least one of a cable 210 (as shown in the illustrated example), wireless technology, etc. For a cable, the complementary interfaces include complementary mechanical components.

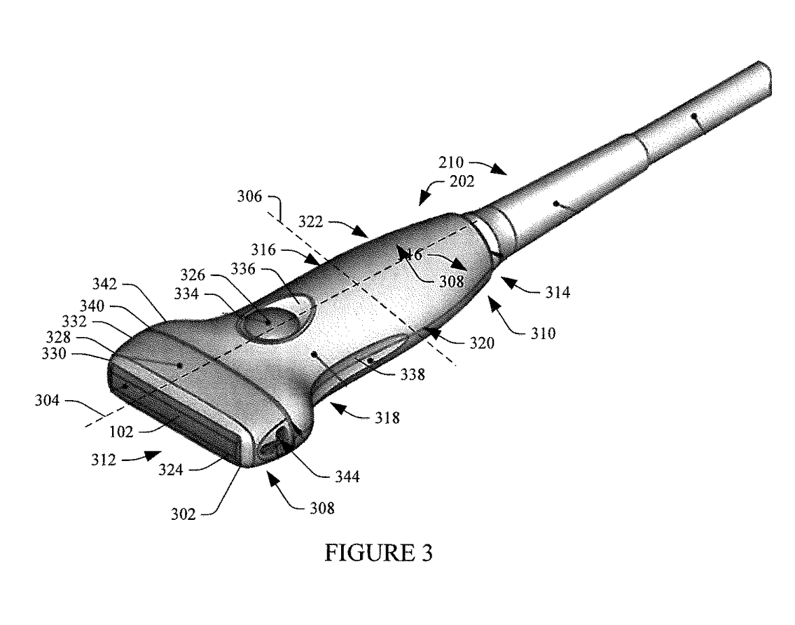

FIG. 3 illustrates an example configuration of the probe 202 and a sub-portion of the cable 210. The probe 202 includes a single enclosure elongate housing 302. The housing 302 has a long axis 304 and a short axis 306, which is transverse to the long axis 304. The housing 302 includes a first end region 308 at one end of the longitudinal axis 304 and a second end region 310 (which opposes the first end region 308) at the opposing end of the longitudinal axis 304.

The housing 302 includes a plurality of sides, which are referred to herein as a front 312, a back 314, a top 316, a bottom 318, a left 320, and a right 322 for distinguishing the sides from each other, but can be referred to otherwise. The front and back sides 312 and 314 oppose each other; the top and bottom sides 316 and 318 oppose each other, and the left and right sides 320 and 322 oppose each other. The left, right, top and bottom sides 320, 322, 316 and 318 extend between the top and bottom sides 316 and 318, forming volumetric cavity there within, which supports and/or encloses at least the transducer array 102 and other electrical and/or mechanical components.

The housing 302 further includes an acoustic window 324 disposed at the front side 312. In one instance, the acoustic window 324 is integrated, e.g., in a cut out or recess, and is part of the front side 312 and/or the housing 302. The transducer array 102 is behind the acoustic window 324, with a transducing surface of the transducer array 102 facing the acoustic window 324. The transducing surface emits and/or receives ultrasound through the acoustic window 324. A control button 326 is configured to control at least an ultrasound imaging function. The control 326 is likewise integrated in the housing 302. In a variation, the control 326 is omitted.

The ultrasound imaging apparatus 100 includes regions that can receive a contaminate. For example, a contaminate may enter a region 328 between a support 330 and a head 332, a region 334 between the control button 326 and the top side 316, a recessed region 336 of the top side 316 in which the control 326 resides, a region 338 which defines in recess in the left side 320 (or a corresponding recess in the right side 322), a region 340 between the head 332 and a body 342, a region 344 that includes a recess in the side 320, and/or other region. In general, these regions include split lines, small details, etc. where traditional wiping with a fabric or cloth with a cleaner is difficult or not possible.

The illustrated housing 302 further includes an antibacterial and/or antimicrobial agent or substance at least on components that face the patient and/or the user. This includes an antibacterial and/or antimicrobial substance in connection with the acoustic window 324, the sides 312-322, and/or the cable 210. An antimicrobial is an agent that kills microorganisms or inhibits their growth. For example, an antimicrobial agent includes disinfectants that kill microbes on non-living surfaces to prevent the spread of illness. An antibacterial is a type of antimicrobial that may kill, inhibit, slow down, stall, etc. the growth of pathogenic bacteria, microbes and/or fungi.

The housing 302, in one instance, includes a polymer with the antibacterial and/or antimicrobial agent at least in and/or on components that face the patient and/or the user. In one instance, the polymer is infused with nano-particles of an element(s) with biocompatible antibacterial and/or antimicrobial properties. For example, a resin with an embedded antibacterial and/or antimicrobial additive can be added to the polymer. A suitable resin is a commercially available resin. Examples of such resins include Xenoy™ LNP67001XXH AM, LEXAN™ EXL LNP D7001 XH AM, and LTL ColorRX PC-1000RX. One such resin includes silver (Ag), silver-titanium oxide (Ag—TiO), copper (Cu), etc. based nanoparticles as the active additive.

In another instance, the polymer (e.g., the sides 312-322 of the housing 302) is coated (e.g., a conformal coating) with the nano-particles of an element(s) with biocompatible antibacterial and/or antimicrobial properties. The antibacterial layer/coating can be applied through painting, dipping, spraying, physical vapor deposition, vacuum arc deposition, sputtering, etc. The active substance of the layer/coating can include Ag, Ag—TiO, Cu, etc. based nanoparticles as the active additive. In general, any biocompatible agent with antibacterial and/or antimicrobial properties can be used.

In one instance, the antibacterial and/or antimicrobial of the housing 202 facilities killing and/or inhibiting the growth of contaminates on the housing 302. This includes contaminates in the regions 328, 334, 336, 338, 340, 344 and/or other regions in which a contaminate may enter and not be removed with fabric or wipe that includes a cleaner, a disinfectant, etc. As such, the antibacterial and/or antimicrobial of the housing 302 may facilitate mitigating cross contamination between patients, where the probe 302 is used with two patients where the first patient is contaminated with a pathogenic bacteria, microbes and/or fungi. A fabric, wipe, etc. may be used in conjunction with the antibacterial and/or antimicrobial.

The probe 202, in between patients, may optionally be placed in an ultraviolet light box that includes a source of ultraviolet light and a support. The source is disposed in the box such that an emitter of the ultraviolet light emits the ultraviolet light towards the support, and the support is configured to support the probe 202 so that at least a predetermined region of interest of the probe 202 (e.g., at least the window 324, etc.) is exposed to the ultraviolet light. The exposure to the ultraviolet light enhances the antimicrobial effect of any silver on the surfaces 212-230 of the housing 302. In general, silver has a relative low redox potential, being easily reduce to metal particles in the present of the ultraviolet light. The ultraviolet light box can be portable and brought to each patient along with the probe 202 or fixed at a location.

In the illustrated embodiment, the cable 210 is fixedly attached to the housing 302. In a variation, the housing 302 and the cable 210 include complementary connectors configured to mechanically and electrically engage and create an electrical path there between. In either instance, the antibacterial and/or antimicrobial can also be embedded in a material of the cable 210, e.g., as a resin additive, an applied layer, etc., similar to the antibacterial and/or antimicrobial on the housing 302. The other end of the cable likewise includes a connector complementary to a connector of the console 204.

Additionally or alternatively, the display 120, the console 204 (including, e.g., the keyboard, touch pad, keypad, trackball, etc.), the mobile cart 206, and/or other components of the ultrasound imaging apparatus 100 can similarly include an antibacterial and/or antimicrobial agent or substance at least on a component that contacts the patient and/or the user.

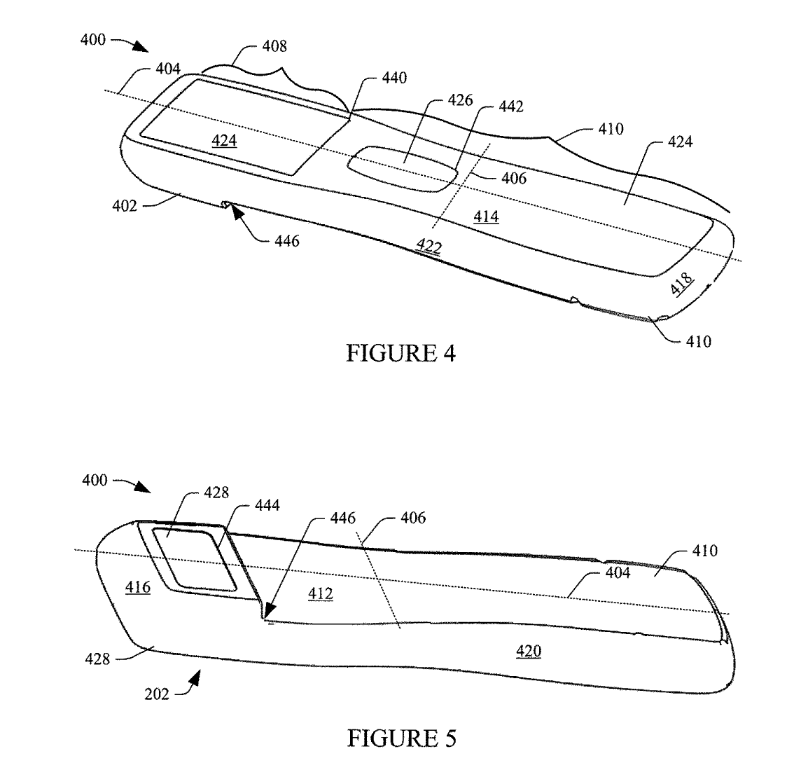

FIGS. 4 and 5 illustrate a variation in which the ultrasound imaging apparatus 100 is a single enclosure elongate hand held ultrasound imaging scanner 400.

In this example, the transducer array 102, the transmit and receive circuitry 106 and 108, the controller, 110, the UI 112, the beamformer 114, the scan converter 116, the rendering engine 118 and the display 120 are included, supported and/or integrated with a housing 402. The housing 402 has a long axis 404 and a short axis 406, which is transverse to the long axis 404. The housing 402 includes a first end region 408 at one end of the longitudinal axis 404 and a second end region 410 (which opposes the first end region 408) at the opposing end of the longitudinal axis 404.

The housing 402 includes a plurality of sides, which are referred to herein as a front 412, a back 414, a top 416, a bottom 418, a left 420, and a right 422 for distinguishing the sides from each other, but can be referred to otherwise. The front and back sides 412 and 414 oppose each other; the top and bottom sides 416 and 418 oppose each other, and the left and right sides 420 and 422 oppose each other. The left and right sides 420 and 422 respectively extend between the top and bottom sides 416 and 418, and the left, right, top and bottom sides 420, 422, 416 and 418 are between front and back sides 412 and 414.

FIG. 4 illustrates a perspective view showing the back, right and bottom sides 414, 422 and 418. The back 414 includes a display screen 424 configured to visually present ultrasound imaging and/or other data. The display screen 424 is integrated in the back 414, e.g., in a cut out or recess therein, and is part of the back 414 and/or the single housing 402. A control 426 is configured to control at least an ultrasound imaging function of the ultrasound imaging scanner 400. The control 426 is integrated in the back 414, e.g., in a cut out or recess therein, and is part of the back 414 and/or the housing 402.

FIG. 5 illustrates a perspective view showing the front, the top and the left sides 412, 416 and 420. The front 412 includes an acoustic window 428, which is integrated in the front 412, e.g., in yet another cut out or recess, and is part of the front 412 and/or the housing 402. In FIGS. 4 and 5, the second end region 408 represents a handle portion of the ultrasound imaging scanner 400. An example of a hand held is described in U.S. Pat. No. 7,699,776, entitled “Intuitive Ultrasonic Imaging System and Related Method Thereof,” and filed on Mar. 6, 2003, which is incorporated herein in its entirety by reference.

Similar to the housing 302, the housing 402 includes an antibacterial and/or antimicrobial substance at least on components that face the patient and/or the user. This includes an antibacterial and/or antimicrobial substance in connection with the acoustic window 428, the sides 412-422. Also similar to the housing 302, the housing 402 may include a polymer with an antibacterial and/or antimicrobial substance at least on components that face the patient and/or the user. Likewise, the polymer can be is infused or coated with nano-particles of an element(s) with biocompatible antibacterial and/or antimicrobial properties such as Ag, Ag—TiO, (Cu), etc.

The ultrasound imaging scanner 400 includes regions that can receive a contaminate. For example, a contaminate may enter a region 440 between the display 424 and the back side 414, a region 442 between the control 426 and the back side 414, a region 444 between the window 428 and the front side 416, at a region 446 of the front side 416. Similarly, the antibacterial and/or antimicrobial of the housing 402 facilities removal of contaminates from the housing 402 and thus facilitates mitigating cross contamination between patients and can be used with other cleaners, disinfectants, etc. Furthermore, the ultrasound imaging scanner 400 can be placed in the ultraviolet light box and exposed to ultraviolet light.

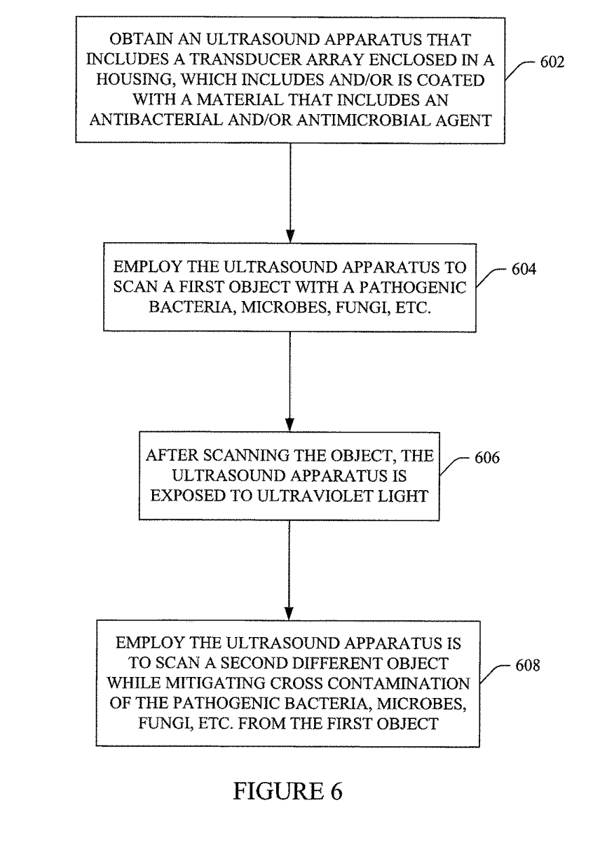

FIG. 6 illustrates a method.

It is to be appreciated that the order of the following acts is provided for explanatory purposes and is not limiting. As such, one or more of the following acts may occur in a different order. Furthermore, one or more of the following acts may be omitted and/or one or more additional acts may be added.

At 602, an ultrasound imaging apparatus with a transducer array enclosed in a housing, which includes and/or is coated with a material that includes an antibacterial and/or antimicrobial agent, is obtained.

At 604, the ultrasound imaging apparatus is employed to scan a first object with a pathogenic bacteria, microbes, fungi, etc. As discussed herein, the antibacterial and/or antimicrobial kills and/or inhibits the pathogenic bacteria, microbes, fungi, etc.

At 606, after scanning the object, the ultrasound imaging apparatus is exposed to ultraviolet light. As discussed herein, this may enhance the effect of the antibacterial and/or antimicrobial kills on the pathogenic bacteria, microbes, fungi, etc. In a variation, this act is omitted.

At 608, the ultrasound imaging apparatus is employed to scan a second different object. As discussed herein, by killing and/or inhibiting the pathogenic bacteria, microbes, fungi, etc., the antibacterial and/or antimicrobial facilitates mitigating cross contamination between the first and second objects.

The application has been described with reference to various embodiments. Modifications and alterations will occur to others upon reading the application. It is intended that the invention be construed as including all such modifications and alterations, including insofar as they come within the scope of the appended claims and the equivalents thereof.

What is claimed is:

1. An ultrasound imaging apparatus, comprising:
a housing, including:
a plurality of sides that include a material with at least one of an antibacterial or an antimicrobial agent thereon;
an acoustic window of one of the sides; and
a transducer array of transducing elements disposed within the sides and adjacent to the acoustic window.

2. The ultrasound imaging apparatus of claim 1, wherein the at least one of an antibacterial or an antimicrobial agent includes at least on of silver, silver-titanium oxide, or copper nano-particles.

3. The ultrasound imaging apparatus of claim 2, wherein the material includes a polymer.

4. The ultrasound imaging apparatus of claim 1, wherein the at least one of an antibacterial or an antimicrobial agent includes a resin embedded with at least one of the antibacterial or an antimicrobial agent.

5. The ultrasound imaging apparatus of claim 1, wherein the material is infused with at least one of silver, silver-titanium oxide, or copper nano-particles.

6. The ultrasound imaging apparatus of claim 1, wherein the plurality of sides includes a coating, which includes at least one of the antibacterial or the antimicrobial agent.

7. The ultrasound imaging apparatus of claim 6, wherein the coating includes an active ingredient from a group consisting of silver, silver-titanium oxide, or copper.

8. The ultrasound imaging apparatus of claim 1, wherein the acoustic window includes at least one of the antibacterial or the antimicrobial agent.

9. The ultrasound imaging apparatus of claim 1, further comprising:
a probe, wherein the housing houses the probe.

10. The ultrasound imaging apparatus of claim 9, further comprising:
a console, which is a separate and distinct device from the probe, the console including:
transmit circuitry configured to transmit control signals to the transducer elements;
receive circuitry configured to receive echo signals to the transducer elements; and
a beamformer that processes the receive echo signals and produces ultrasound images.

11. The ultrasound imaging apparatus of claim 10, the probe including a first connector, and the console including a second connector, and further comprising: at least one of a wireless communications interface or a cable configured to connect to the probe and the console, thereby establishing an electrical communications path there between.

12. The ultrasound imaging apparatus of claim 10, further comprising:
a cart, wherein the console is affixed to the mobile cart.

13. The ultrasound imaging apparatus of claim 1, further comprising:
a single elongate hand held housing, including:
transmit circuitry configured to transmit control signals to the transducer elements;
receive circuitry configured to receive echo signals to the transducer elements;
a beamformer that processes the receive echo signals and produces an ultrasound image;
a display configured to display an ultrasound image; and
a control configured to control at least one ultrasound function.

14. An ultrasound imaging apparatus, comprising:
a probe with a surface with an antibacterial or an antimicrobial integrated thereon, the probe enclosing a transducer array and including a first communications interface, wherein the surface of the probe includes at least one recess; and
a console, including: transmit circuitry configured to transmit control signals to the transducer elements, receive circuitry configured to receive echo signals to the transducer elements, a beamformer that processes the receive echo signals and produces ultrasound images, and a complementary communications interface.

15. The ultrasound imaging apparatus of claim 14, wherein the at least one of an antibacterial or an antimicrobial agent is from a group consisting of silver, silver-titanium oxide, or copper nano-particles.

16. The ultrasound imaging apparatus of claim 15, wherein the at least one of an antibacterial or an antimicrobial agent is included in the surface.

17. The ultrasound imaging apparatus of claim 15, wherein the at least one of an antibacterial or an antimicrobial agent is a layer on the surface.

18. A single enclosure hand held ultrasound imaging apparatus, comprising:
a housing with a surface having an antibacterial or an antimicrobial coating therein, the housing enclosing a transducer array, transmit circuitry, receive circuitry, a beamformer, a control and a display.

19. The single enclosure hand held ultrasound imaging apparatus of claim 18, wherein the at least one of an antibacterial or an antimicrobial agent is from a group consisting of silver, silver-titanium oxide, or copper nano-particles.

20. The single enclosure hand held ultrasound imaging apparatus of claim 15, wherein the at least one of an antibacterial or an antimicrobial agent is at least one of included in the surface or in a coating on the surface.
